(19) United States
(12) Patent Application Publication
(10) Pub. No.: US 2010/0062046 A1
(43) Pub. Date: Mar. 11, 2010

(54) POLYMORPHS OF NICOTINIC INTERMEDIATES

(76) Inventors: Douglas J.M. Allen, New London, CT (US); Travis L. Houston, Lafaayette, IN (US); Lien H. Koztecki, Indianapolis, IN (US); Melissa J. Casteel, Norwich, CT (US); David Burns Damon, Mystic, CT (US)

Correspondence Address: PFIZER INC;Mary J Hosley, 150 EAST 42ND STREET, MS: 150/02/E112, NEW YORK, NY, 10017-5612, US

(21) Appl. No.: 12/447,516

(22) PCT Filed: Nov. 9, 2007
(86) PCT No.: PCT/US07/23683

§ 371 Date: Apr. 28, 2009

Related U.S. Application Data
(60) Provisional application No. 60/865,077, filed on Nov. 9, 2006.
(60) Provisional application No. 60/985,406, filed on Nov. 5, 2007.

(51) Int. Cl. A61K 31/4985; C07D 471/04; A61K 9/70

(52) U.S. Cl. 424/449; 544/343; 514/250

(57) ABSTRACT

Crystalline forms of compounds (II), (III) and (IV) and processes to produce them are provided.

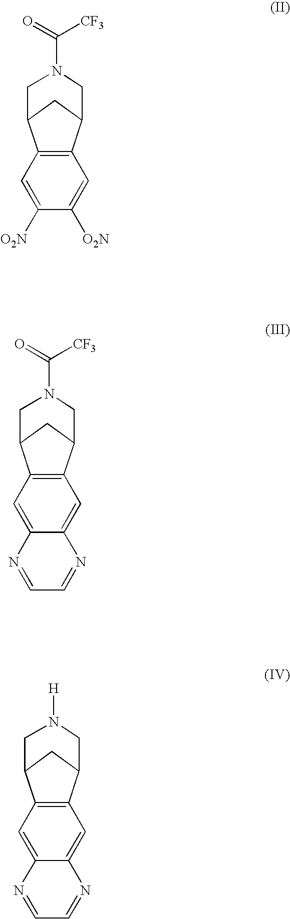

### FIELD OF THE INVENTION

This invention relates to crystal forms of intermediates used in the process to prepare varenicline tartrate including the varenicline free base.

### BACKGROUND OF THE INVENTION

Varenicline tartrate (V) is an FDA approved drug for use in facilitating smoking cessation. Compounds I-IV are intermediates in the synthesis of V.

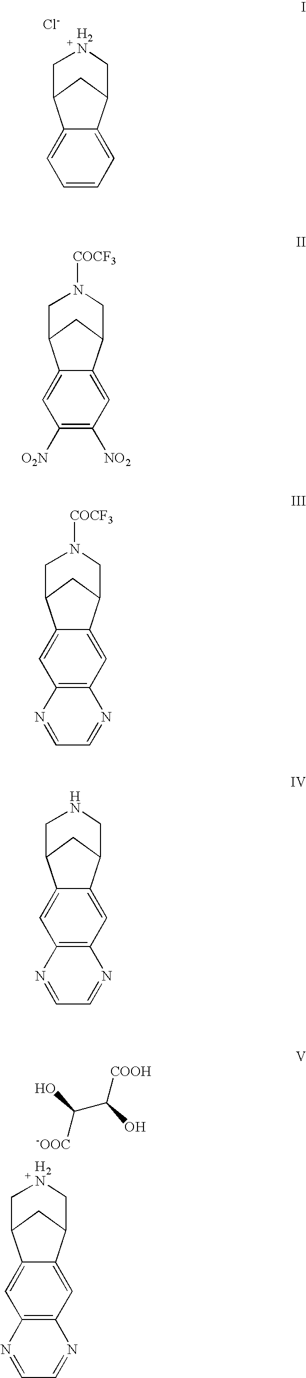

Varenicline tartrate (V) has been isolated and characterized in U.S. Pat. No. 6,890,925. The intermediates (I, II and III) and the free base of varenicline (IV) have been isolated and generically characterized in U.S. Pat. No. 6,410,550. The disclosures of these patents are incorporated herein by reference thereto.

The intermediate compound I is known and identified as:

| | |
|---|---|
| 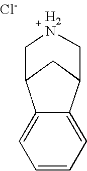 | CAS Name: 1,5-Methano-1H-3-benzazepine- 2,3,4,5-tetrahydro-,hydrochloride CAS Number: 230615-52-8 Molecular Formula: C₁₁H₁₃N•HCl Molecular Weight: 195.69 |

The intermediate compound II is known and identified as:

| | |
|---|---|
| 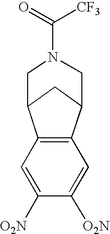 | CAS Name: 1,5-Methano-1H-3-benzazepine- 2,3,4,5-tetrahydro-7,8-dinitro-3-(trifluoroacetyl) CAS Number: 230615-59-5 Molecular Formula: C₁₃H₁₀F₃N₃O₅ Molecular Weight: 345.23 |

Intermediate compound III is known and identified as:

| | |
|---|---|
| 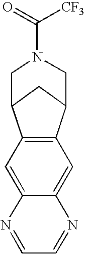 | CAS Name: 6,10-Methano-6H-pyrazino[2,3- h][3]benzazepine,7,8,9,10-tetrahydro-8- (trifluoroacetyl) CAS Number: 230615-70-0 Molecular Formula: C₁₅H₁₂F₃N₃O Molecular Weight: 307.27 |

The free base of varenicline, intermediate compound IV is known and identified as:

| | |
|---|---|
| 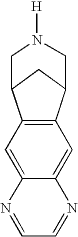 | Chemical name: 6,7,8,9-tetrahydro-6H- pyrazino[2,3,-h][3]benzazepine Chemical formula: C₁₃H₁₃N₃ Molecular weight: 211.26 |

It has been discovered that the isolated compounds of formulas II and III as well as the formula IV, free base varenicline, exist in crystalline form states which have not been previously synthesized, isolated or even characterized.

Generally, the present invention comprises previously unknown, and uncharacterized, crystalline forms of compounds II, III and IV, individually and/or in combination with each other or previously isolated but not characterized crystalline forms. The starting material of compound I has, as far as has been determined, only been characterized in a single crystalline form but compounds II, III and IV have each been discovered to exist in at least two distinct crystalline forms (compounds II and III) or at least four distinct crystalline forms (compound IV).

### SUMMARY OF THE INVENTION

It is an object of the present invention to provide crystalline forms of the intermediate compounds I-IV.

It is a further object of the present invention to provide crystalline forms of the intermediate compounds II, III and IV which have not been previously synthesized, isolated or characterized.

It is a further object of the present invention to provide such crystalline forms in essentially pure form and/or in admixture with crystalline forms inherently made by prior art processes but not characterized as isolated crystalline forms.

It is a further object of the present invention to provide methods for the production of such crystalline forms with specific characterization identification.

It is a further object of the present invention to provide a composition comprising substantially pure varenicline free base Form C suitable for administration to a human subject comprising less than 2% by weight of N-formylvarenicline adduct relative to the total weight of varenicline and less than 2% by weight of N-carboxyvarenicline adduct relative to the total weight of varenicline.

It is a further object of the present invention to provide a composition of varenicline in a transdermal patch wherein the substantially pure varenicline free base Form C is a particulate suspension.

It is a further object of the present invention to provide a process to form substantially pure varenicline free base form C suitable for administration to a human subject comprising a) less than 2% by weight of N-formylvarenicline, and b) less than 2% by weight of N-carboxyvarenicline adduct, comprising the step of crystallizing varenicline from the crystallization solvent or solvent combination comprising an organic non-chlorinated solvent.

It is a further object of the present invention to provide a process wherein the crystallization solvent or solvent combinations used to isolate substantially pure varenicline free base form C comprises an organic non-chlorinated solvent.

It is a further object of the present invention to provide a process wherein said non-chlorinated solvent or solvent combinations selected from the group consisting of toluene, xylenes, hexanes, cyclohexanes, heptanes, n-heptane, octanes, nonanes and decanes.

It is a further object of the present invention to provide a process further comprising a seeding step to prepare smaller sized particles of substantially pure varenicline free base form C.

These and other objects, features and advantages of the present invention will become more evident from the following discussion and drawings.

### BRIEF DESCRIPTION OF THE DRAWINGS

FIG. 1 is an x-ray powder diffraction pattern of Form A of compound I.

FIGS. 2a and 2b are x-ray powder diffraction patterns of Forms A and B of compound II, respectively.

FIGS. 3a and 3b are x-ray powder diffraction patterns of Form A and Form A+B of compound III, respectively.

FIG. 4 is an x-ray powder diffraction pattern of Form A of compound IV (varenicline free base).

FIG. 5 is a process scheme to produce Form C of compound IV (varenicline free base).

FIGS. 6a, 6b, 6c, and 6d are an x-ray powder pattern diffraction pattern of Form C of Compound IV (varenicline free base).

FIG. 7 is a calculated x-ray powder pattern diffraction pattern of Form D of compound IV (varenicline free base).

FIG. 8 is an x-ray powder pattern diffraction pattern of Form E of compound IV (varenicline free base).

FIG. 9 is a FT-IR ATR spectrum of Form C of compound IV (varenicline free base).

FIG. 10 is a FT-Raman spectrum of Form C of compound IV (varenicline free base).

FIG. 11 is a ¹³C CPMAS spectrum of Form C of compound IV (varenicline free base).

FIG. 12 is an x-ray powder pattern diffraction pattern of the N-carboxyvarenicline adduct.

FIG. 13 is a FT-Raman of the N-carboxyvarenicline adduct.

FIG. 14 is a calculated x-ray powder pattern diffraction pattern of N-formylvarenicline.

### DETAILED DESCRIPTION OF THE INVENTION

### Crystalline Form Forms of Compound I

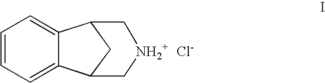

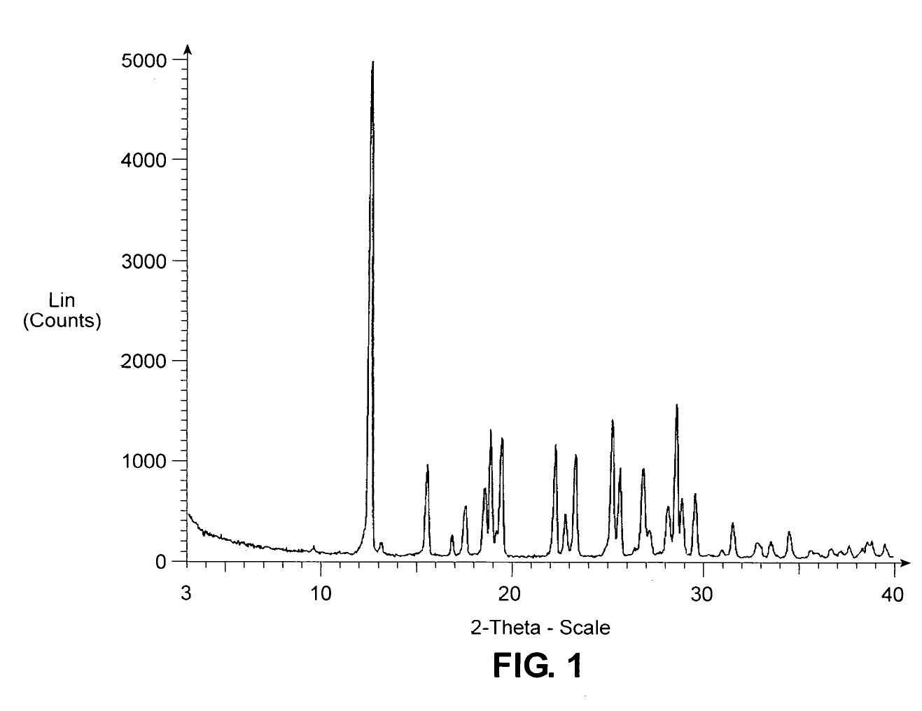

Compound I has, as far as has been determined, only been characterized in a single crystalline form, Form A. The x-ray powder diffraction pattern of Form A of compound I is provided in FIG. 1.

The X-ray powder diffraction pattern was generated with a Siemens D5000 diffractometer using CuK α radiation. The instrument was equipped with a line focus X-ray tube. The tube voltage and amperage were set to 40 kV and 30 mA, respectively. The divergence and scattering slits were set at 1 mm, and the receiving slit was set at 0.6 mm. Diffracted CuKα1 radiation (λ=1.54056 Å) was detected using a Sol-X energy dispersive X-ray detector. A theta two theta continuous scan at 2.4°2θ/min (1 sec/0.04°2θ step) from 3.0 to 40°2θ was used. An alumina standard (NIST standard reference material 1976) was analyzed to check the instrument alignment. Data were collected and analyzed using BRUKER AXS DIFFRAC PLUS software Version 2.0. Samples were prepared for analysis by placing them in a quartz holder. The sample powder was pressed by a glass slide or equivalent to ensure a random surface and proper sample height. The sample holder was then placed into the Bruker instrument and the powder x-ray diffraction pattern was collected using the instrumental parameters specified above. Measurement differences associated with such X-ray powder diffraction analyses result from a variety of factors including: (a) errors in sample preparation (e.g., sample height), (b) instrument errors, (c) calibration errors, (d) operator errors (including those errors present when determining the peak locations), and (e) the nature of the material (e.g. preferred orientation errors). Calibration errors and sample height errors often result in a shift of all the peaks in the same direction. Small differences in sample height when using a flat holder will lead to large displacements in x-ray powder diffraction peak positions. A systematic study showed that a sample height difference of 1 mm could lead to peak shifts as high as 1°2-theta (Chen et al.; J Pharmaceutical and Biomedical Analysis, 2001; 26, 63). These shifts can be identified from the X-ray diffractogram and can be eliminated by compensating for the shift (applying a systematic correction factor to all peak position values) or recalibrating the instrument. As mentioned above, it is possible to rectify differences in measurements from the various instruments by applying a systematic correction factor to bring the peak positions into agreement.

### Crystalline Form of Compound II

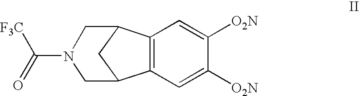

Compound II has been determined to have at least two crystalline forms, with the two being designated Form A and Form B.

Form A was obtained by evaporating or slurrying compound II in solvent systems such as isopropyl alcohol, methanol, THF, water, water/acetonitrile under a variety of temperature conditions.

Form B was obtained by a procedure which encompasses organic solvent slurries, fast evaporation, and slow cooling of filtrates from the saturated solutions. Crystallization included rapid cooling of saturated solutions (crash cools) and rapid precipitation by antisolvent addition (solvent/antisolvent crystallization).

Form B was obtained mainly from fast evaporations of ethyl acetate and methyl ethyl ketone, and from a solvent/antisolvent. Studies conducted in dichloromethane, ethyl acetate, methanol, and toluene indicated that Form A is more stable than Form B at ambient temperature and 60° C. Form A has a melting point of ˜177° C. and Form B has a melting point of ˜170° C.

Form A was determined to have a monoclinic crystal system with a P21/c space group. The unit cell parameters are: a=9.6 Å, b=7.7 Å, c=18.7 Å, α=γ=90°, β=96.9°, cell volume=1381.8 Å³.

Form B is determined to have a triclinic crystal system with a P-1 space group. The cell parameters are: a=8.2 Å, b=9.5 Å, c=9.8 Å, α=81.4°, β=80.6°, γ=67.8°, cell volume=697.4 Å³.

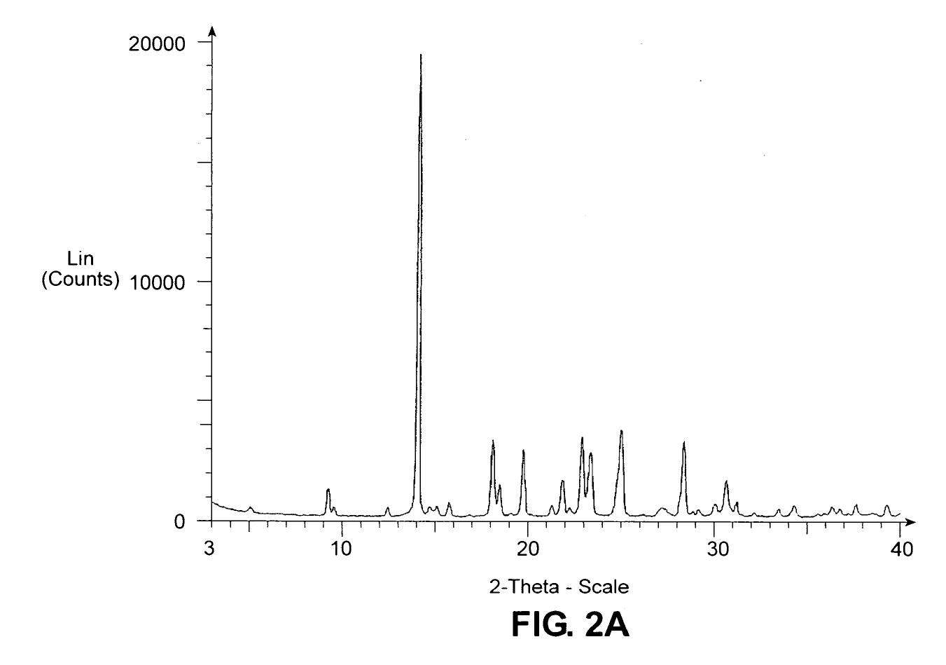

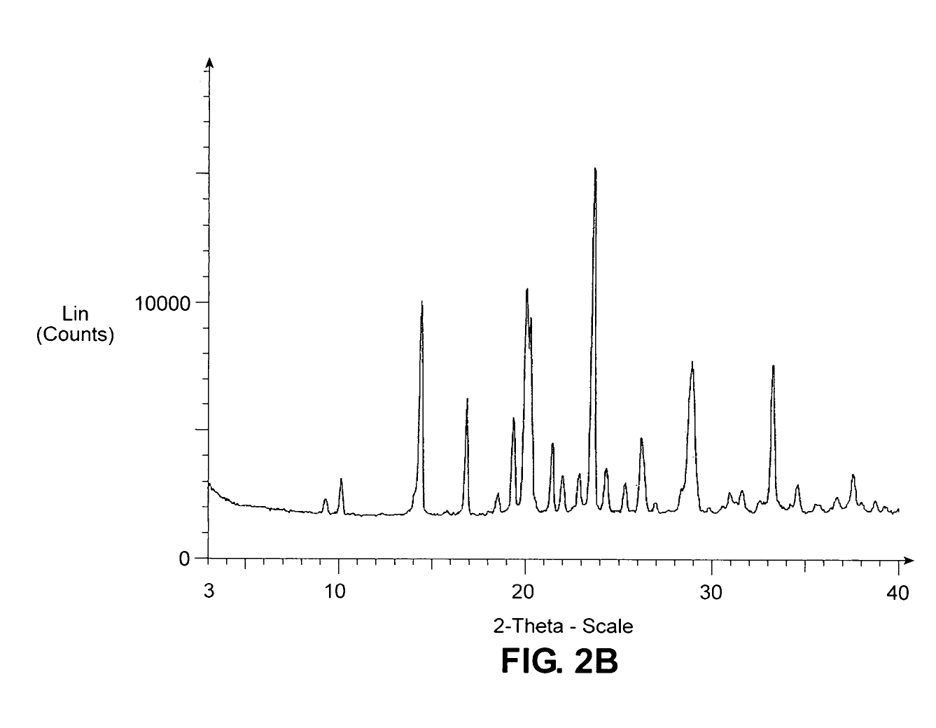

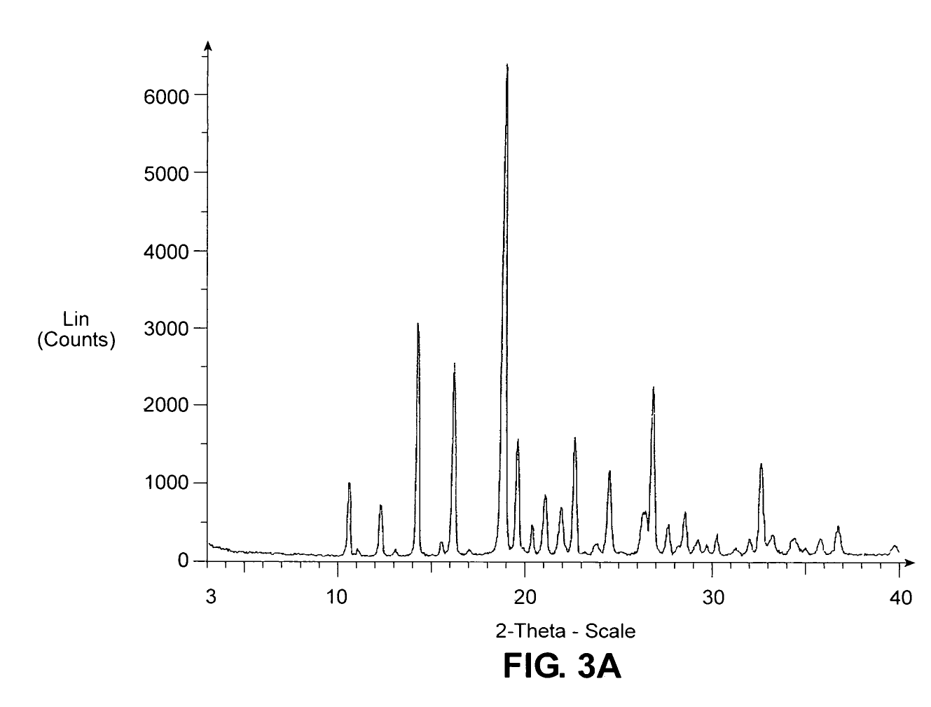

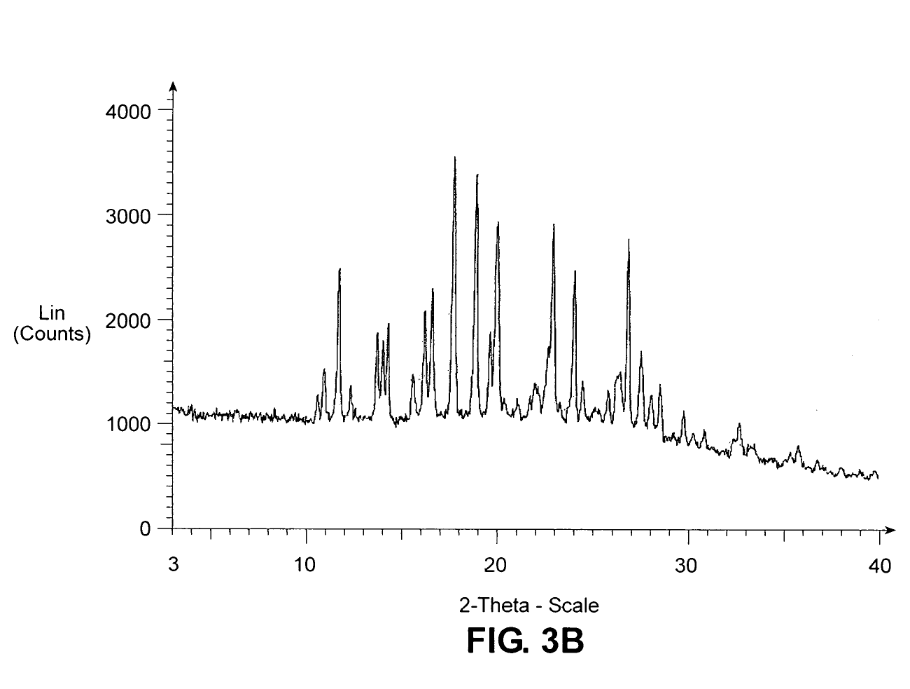

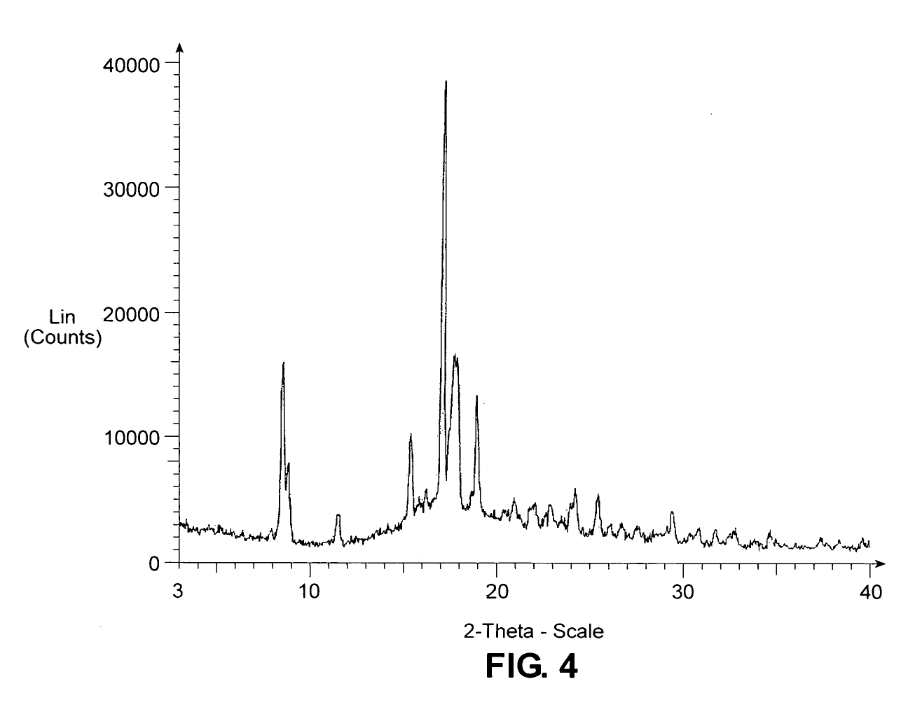

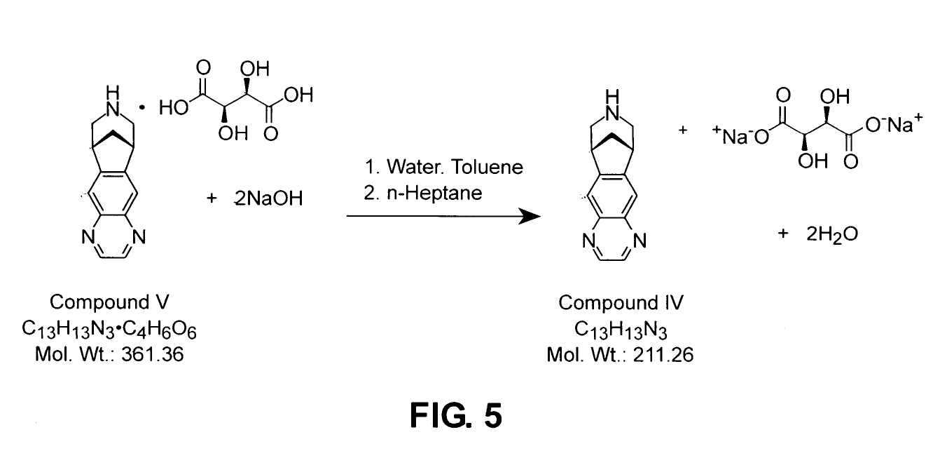

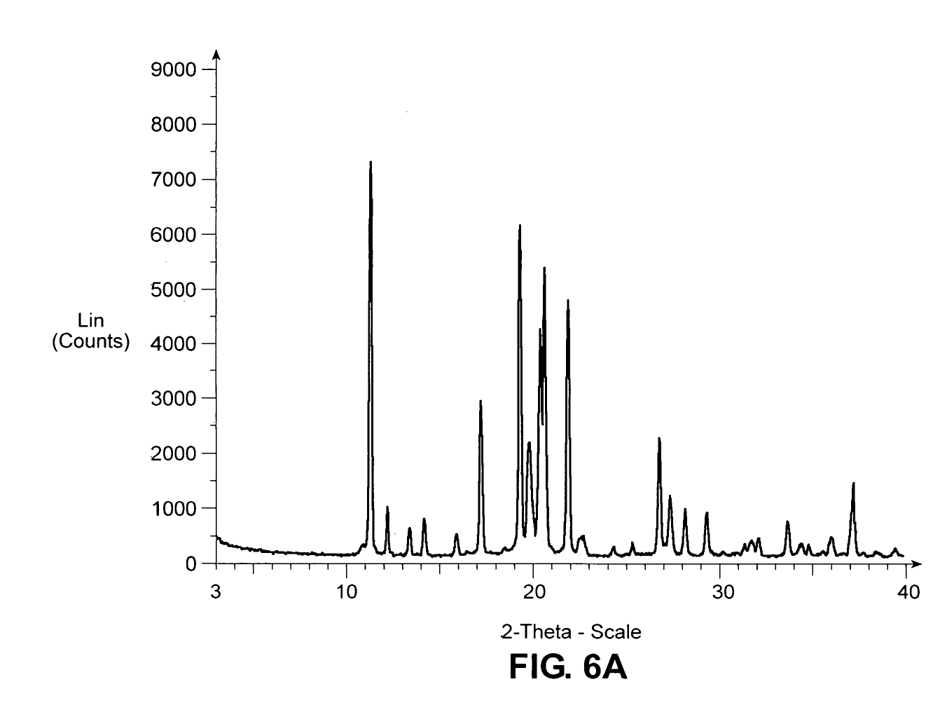

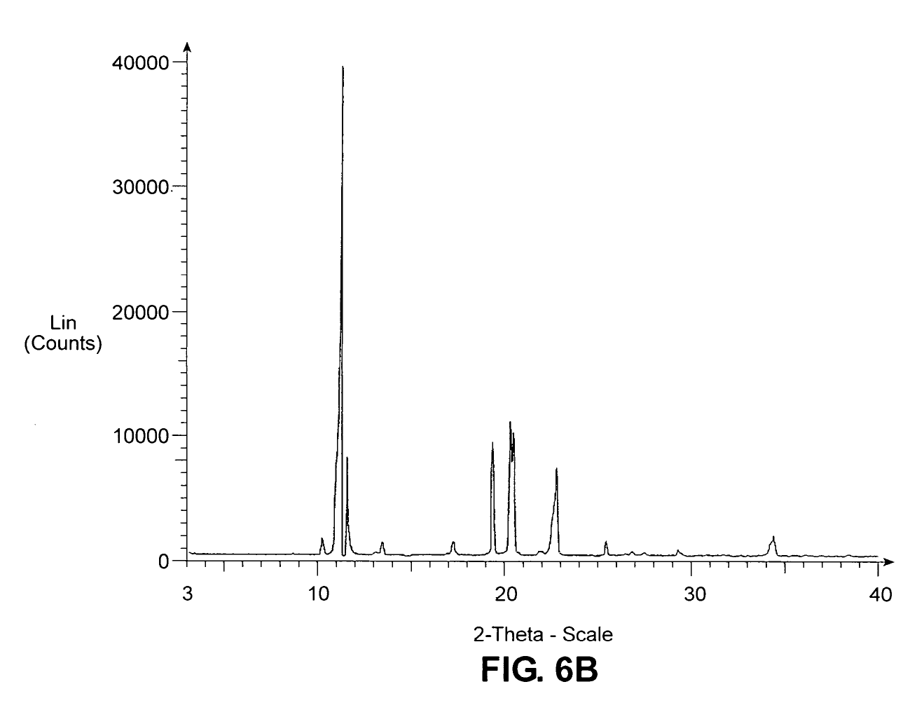

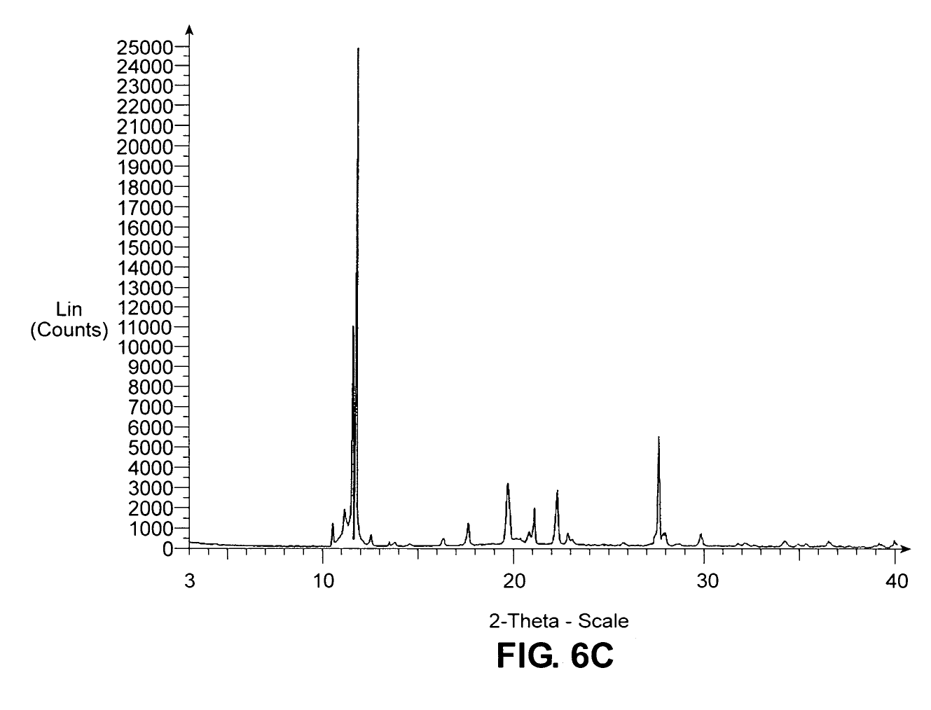

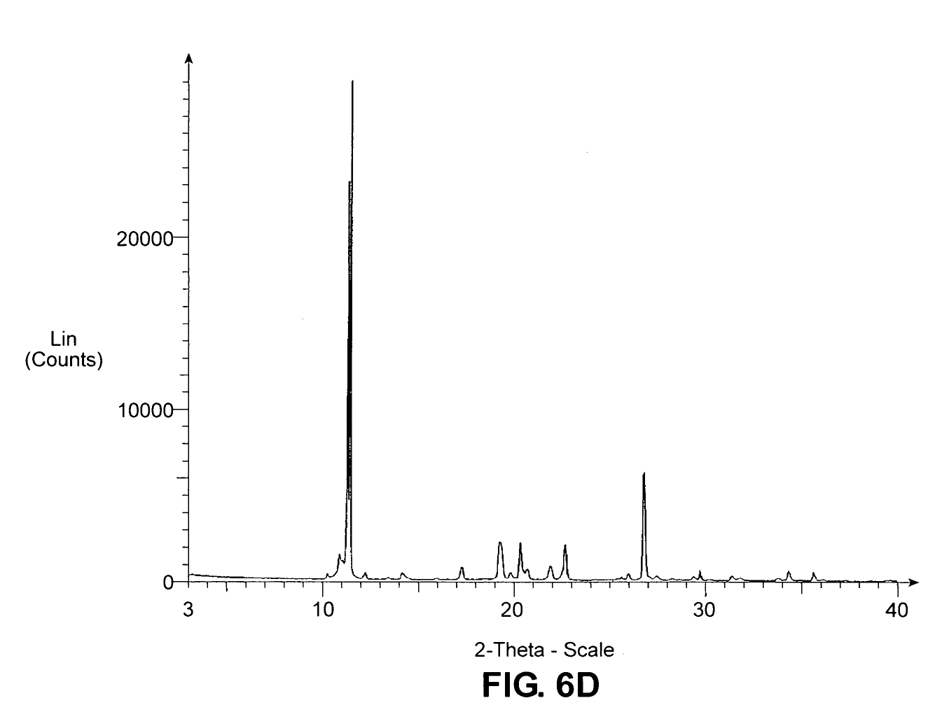

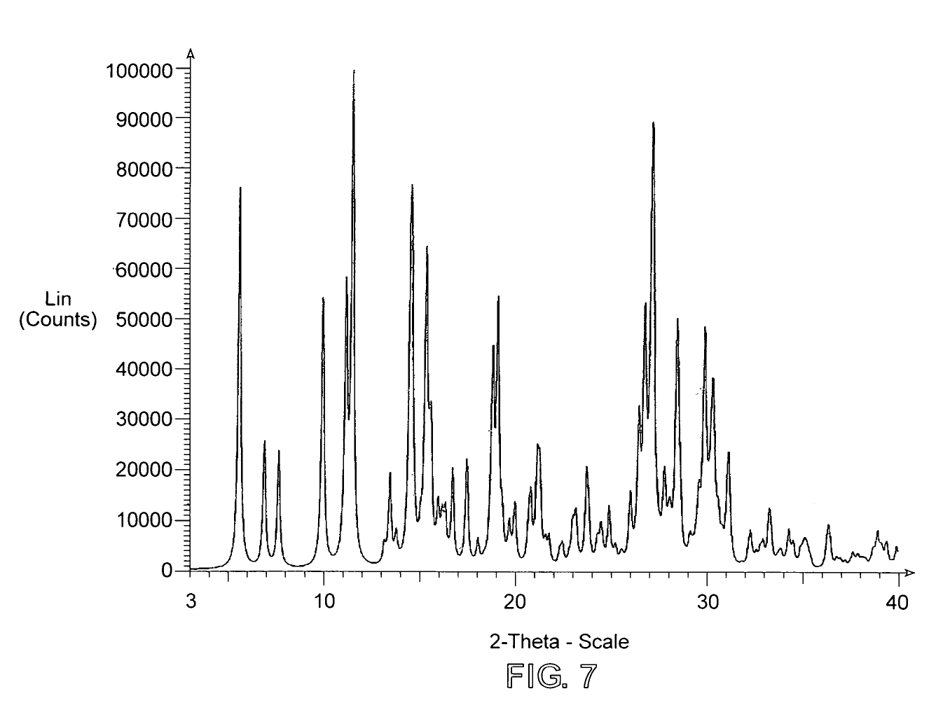

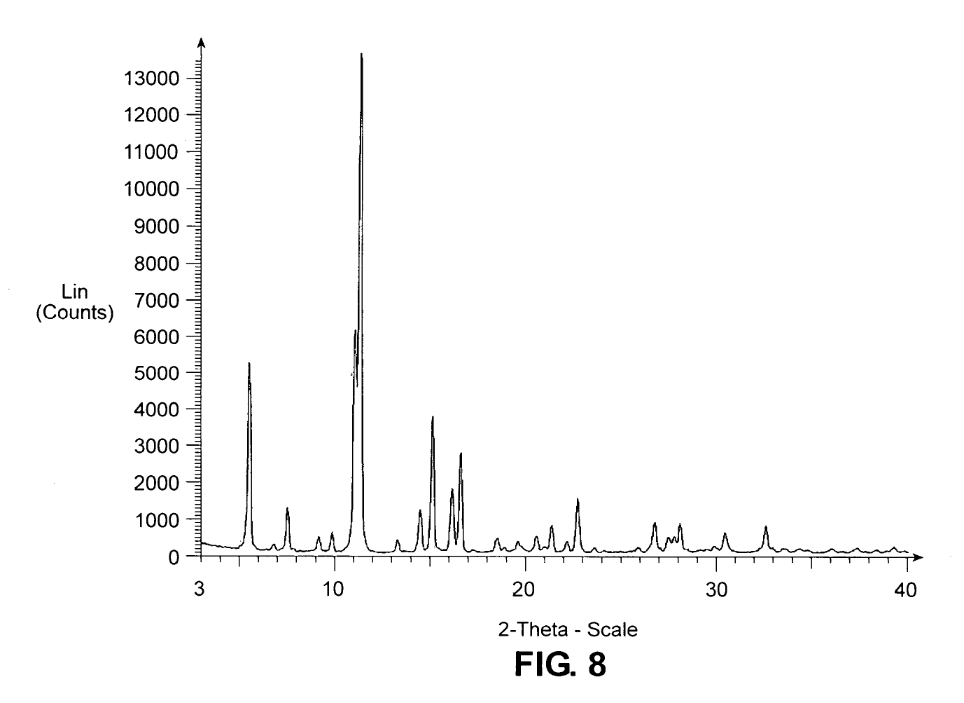

Table 1 is a tabular comparison of x-ray powder diffraction patterns for Forms A and B (up to approximately 33 degrees two-theta; generated with a Siemens D5000 diffractometer as described above; see FIGS. 2a and 2b). Reflections with relative intensity greater than approximately 2% are included.

TABLE 1

| Form A | Form A | Form B | Form B |
|---|---|---|---|
| 2-theta | Relativity Intensity | 2-theta | Relativity Intensity |
| (degrees) | (%) | (degrees) | (%) |
| 9.2 | 6.5 | 9.2 | 16.9 |
| 9.5 | 2.5 | 10.1 | 3.7 |
| 12.4 | 2.5 | 14.0 | 13.9 |
| 14.0 | 100.0 | 14.3 | 71.9 |
| 14.7 | 2.7 | 15.7 | 3.1 |
| 15.1 | 2.7 | 16.8 | 58.2 |
| 15.8 | 3.6 | 18.5 | 15.8 |
| 18.1 | 17.1 | 19.3 | 22.1 |
| 18.5 | 7.5 | 19.9 | 73.9 |
| 19.8 | 15.0 | 20.2 | 96.8 |
| 21.3 | 3.0 | 21.4 | 14.8 |
| 21.9 | 8.5 | 21.9 | 14.7 |
| 22.3 | 2.5 | 22.8 | 13.3 |
| 22.9 | 17.9 | 23.1 | 8.1 |
| 23.4 | 14.5 | 23.5 | 100.0 |
| 25.0 | 19.5 | 24.3 | 13.7 |
| 27.2 | 2.8 | 25.3 | 12.9 |
| 28.3 | 17.1 | 26.1 | 25.8 |
| 30.0 | 3.5 | 26.3 | 19.3 |
| 30.6 | 8.8 | 28.3 | 9.4 |
| 31.2 | 4.0 | 28.8 | 35.2 |
| | | 30.9 | 7.1 |
| | | 31.2 | 4.3 |
| | | 31.5 | 6.6 |
| | | 32.5 | 4.2 |
| | | 33.1 | 30.6 |

TABLE 2

| Form A | Form B |
|---|---|
| 2-theta | 2-theta |
| (degrees) | (degrees) |
| 9.5 | 10.1 |
| 14.0 | 14.3 |
| 14.7 | 19.3 |
| 15.1 | 20.2 |
| 18.1 | 24.3 |
| 25.0 | 33.1 |

The above identified two crystalline forms of compound II were generated and designated as Form A and Form B. Form A is an anhydrous, non-hygroscopic, crystalline form that has a melt with an onset at approximately 177° C.

Form B is an anhydrous, non-hygroscopic, crystalline form that converts to Form A upon heating. Form A is more stable than Form B at ambient temperature and at 60° C.

Various solvents and conditions were utilized to provide the A and B crystalline forms, either separately or in admixture. Tables 3 and 4 summarize the solvents and conditions, with resultant yields:

TABLE 3

| Solvent | Conditions | Results |
|---|---|---|
| Acetonitrile (ACN) | Fast evaporation | A + B |
| | Slurry | A |
| | Slow Cool | A + B |
| | Crash cool of saturated | A |
| | solutions | |
| Methylene chloride (DCM) | Fast evaporation | A + B |
| | Slurry | A |
| | Slow Cool | A |
| Diethyl ether | Fast evaporation | A + B minor |
| | Slurry | A |
| | Slow cool | A |
| Ethanol (EtOH) | Fast evaporation | A + B |
| | Slurry | A |
| | Slow cool | A + B |
| | Slurry | A |
| | Slow cool | A |
| | Crash cool of saturated | A + B |
| | solution | |
| Methyl ethyl ketone (MEK) | Fast evaporation | B |
| | Slurry | A |
| | Slow cool | A + B minor |
| | Crash cool of saturated | B |
| | solution | |
| Tetrahydrofuran (THF) | Fast evaporation | B + A |
| | Slurry | A |
| | Slow cool | A |
| | Slurry | A |
| | Slurry (60° C.) | A |
| | Slow cool | A + B |
| Isolated from EtOH EtOAc, | Fast evaporation solids | A + B |
| ether, isopropyl alcohol | held at 60° C. | |
| (IPA), MeOH and toluene | | |
| slow cools | | |

TABLE 4

| Solvent | Antisolvent | Results |
|---|---|---|
| ACN | Ether | A + B |
| Methanol (MeOH) | Hexanes and ether | A + B |
| THF | H₂O | B |

### Crystalline Forms of Compound III:

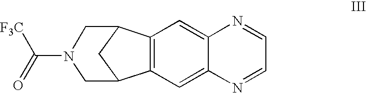

Form A was obtained from the prior art synthesis described in said U.S. Pat. No. 6,410,550. One additional solid-state form was identified during a procedure that encompassed organic solvent slurries, fast evaporation, and slow cooling of filtrates from the saturated solutions. Crystallization included rapid cooling of saturated solutions (crash cools) and rapid precipitation by antisolvent addition (solvent/antisolvent crystallization). The new solid form was generated from solvent/antisolvent evaporation in methanol and isopropyl ether. The solid was determined to be a mixture of the previously known material (Form A, starting material) and a second crystalline material (Form B). Form B was observed in mixtures with Form A but was not isolated as a pure solid phase. Form A appears to be the thermodynamically stable solid-state form.

Form A of compound III is a crystalline, anhydrous, non-hygroscopic solid. Form A+B of compound III is a crystalline, anhydrous, non-hygroscopic solid.

### Crystalline Form Preparation and Procedures

### Analytical

### X-Ray Powder Diffraction (XRPD)

Form A X-ray powder diffraction (XRPD) analyses were performed using a Siemens D5000 diffractometer as described above.

Form A+B X-ray powder diffraction (XRPD) analyses were performed using an Inel XRG-3000 diffractometer equipped with a CPS (Curved Position Sensitive) detector with a 2θ range of 120° . Real time data were collected using CuK α radiation (wavelength 1:1.54056) starting at approximately 4°2θ at a resolution of 0.03°2θ. The tube voltage and amperage were set to 40 kV and 30 mA, respectively. The monochromator slit was set at 5 mm by 160 μm. The pattern is displayed from 2.5 to 40°2θ. Samples were prepared for analysis by packing them into thin-walled glass capillaries. Each capillary was mounted onto a goniometer head that is motorized to permit spinning of the capillary during data acquisition. The samples were analyzed for 5 minutes. Instrument calibration was performed using a silicon reference standard.

### Sample Preparation

### Form A+B

An aliquot of methanol (700 μl) was added to compound III (40 mg). The solution was filtered through a 0.2 μm filter into a vial containing isopropyl ether (1000 μl). Precipitation was not observed. The vial was capped and placed in the hood for one day. No solids were observed. The sample was then placed into a refrigerator for 5 days. After 5 days in the refrigerator, the sample was transferred to a freezer for 8 days. A yellow solution with very fine solids was observed. The solids went into solution as the sample warmed. The vial was then placed in the hood to evaporate under ambient conditions. The resulting solids were dried under vacuum for 3 days.

Two crystalline forms were generated of which one was new. This material was designated as Form B. Form B was obtained from solvent/antisolvent evaporation in methanol and isopropyl ether. Form B was obtained only as a mixture with the previously known Form A. FIGS. 2a and 2b are X-ray powder diffraction patterns of Form A and Form A+B of compound III.

### Characterization

Form A

Crystalline solids generated exhibited XRPD patterns consistent with the starting material were designated as Form A.

Form A+B

A crystalline solid generated from a methanol/IPE antisolvent crystallization exhibited an XRPD pattern similar to Form A with some additional peaks shown in FIG. 2b. This solid material was a mixture of Form A and a new crystalline material “Form B”. The mixture was designated as Form A+B and formed with a solvent/antisolvent (MeOH and IPE) crystallization when the starting material was initially subjected to evaporation. Form A was obtained in the absence of pre-evaporation.

Tables 5, 6 and 7 contain the XRPD peaks greater than approximately 2% relative intensity obtained for Forms A, A+B, and the peaks attributed to B, respectively. Table 8 shows the unique identifying peaks for each of the crystalline forms of Compound III.

TABLE 5

| 2-theta | Relativity Intensity |
|---|---|
| (degrees) | (%) |
| 10.5 | 15.4 |
| 11.0 | 2.1 |
| 12.3 | 11.0 |
| 13.0 | 2.1 |
| 14.2 | 47.4 |
| 15.6 | 3.5 |
| 16.2 | 39.6 |
| 17.1 | 2.0 |
| 18.8 | 100.0 |
| 19.6 | 24.3 |
| 20.4 | 7.1 |
| 21.1 | 13.3 |
| 22.0 | 10.7 |
| 22.7 | 24.7 |
| 23.8 | 3.3 |
| 24.5 | 18.3 |
| 26.4 | 9.8 |
| 26.8 | 35.0 |
| 27.7 | 7.4 |
| 28.2 | 2.9 |
| 28.5 | 9.9 |
| 29.2 | 4.2 |
| 29.8 | 3.5 |
| 30.3 | 5.4 |
| 31.2 | 2.7 |
| 32.0 | 4.5 |
| 32.6 | 19.6 |
| 33.3 | 5.3 |
| 34.4 | 4.8 |
| 35.0 | 2.7 |
| 35.8 | 4.6 |
| 36.8 | 7.3 |

TABLE 6

| 2-theta | Relativity Intensity |
|---|---|
| (degrees) | (%) |
| 10.5 | 35.6 |
| 10.8 | 42.7 |
| 11.0 | 31.1 |
| 11.6 | 69.7 |
| 12.3 | 38.1 |
| 12.5 | 32.0 |
| 13.7 | 52.3 |
| 14.0 | 50.3 |
| 14.2 | 55.1 |
| 15.5 | 41.2 |
| 16.1 | 58.5 |
| 16.5 | 64.6 |
| 17.6 | 100.0 |
| 18.8 | 95.2 |
| 19.6 | 52.7 |
| 19.9 | 81.9 |
| 20.4 | 34.8 |
| 21.0 | 34.8 |
| 21.9 | 38.9 |
| 22.6 | 48.4 |
| 22.9 | 82.0 |
| 23.3 | 33.8 |
| 23.7 | 33.0 |
| 24.0 | 69.5 |
| 24.4 | 39.7 |
| 25.8 | 37.2 |
| 26.4 | 41.8 |
| 26.8 | 78.2 |
| 27.5 | 47.9 |
| 28.0 | 35.8 |
| 28.5 | 38.8 |
| 29.2 | 26.0 |
| 29.7 | 31.0 |
| 30.2 | 25.6 |
| 30.8 | 26.6 |
| 32.4 | 24.0 |
| 32.6 | 28.4 |
| 33.2 | 22.3 |
| 35.0 | 18.9 |
| 35.3 | 20.8 |
| 35.7 | 22.5 |
| 36.7 | 18.5 |

TABLE 7

| 2-theta | Relativity Intensity |
|---|---|
| (degrees) | (%) |
| 10.8 | 42.7 |
| 11.6 | 69.7 |
| 12.5 | 32.0 |
| 13.7 | 52.3 |
| 14.0 | 50.3 |
| 16.5 | 64.6 |
| 17.6 | 100.0 |
| 19.9 | 81.9 |
| 22.9 | 82.0 |
| 23.3 | 33.8 |
| 24.0 | 69.5 |
| 25.8 | 37.2 |
| 30.8 | 26.6 |
| 32.4 | 24.0 |
| 35.3 | 20.8 |

TABLE 8

| Form A | Form B |
|---|---|
| 2-theta | 2-theta |
| (degrees) | (degrees) |
| 10.5 | 11.6 |
| 16.2 | 13.7 |
| 18.8 | 16.5 |
| 19.6 | 17.6 |

### Crystalline Forms of Compound IV

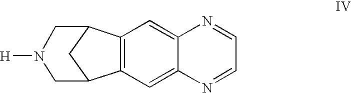

Novel crystalline forms of the free base of varenicline, compound IV, have been discovered. The respective crystalline forms are designated herein as Form A, Form C, Form D, and Form E.

The following methods were used to prepare each of the crystalline forms of varenicline free base:

### Form A

Approximately 1 mg of compound IV (varenicline free base) was heated to form a melt. The melt crystallized between 120-155° C. to form crystals with plate and lath morphologies. These crystals were added to a slurry of crystalline compound IV in ethyl acetate. The slurry was stirred for one hour at ambient conditions. The solid was isolated by filtration.

### Form C

### A. Unseeded Process

Varenicline tartrate (15 g) was dissolved in water (75 mL), then toluene (255 mL) was added. The mixture was heated to approx. 38° C., then 50% NaOH (7.29 g) was added. After 1.5 hours, the mixture was treated with a slurry of activated carbon (0.75 g) in toluene (5 mL), and then filtered through a cake of diatomaceous earth. The filter cake was washed with toluene (22.5 mL).

The filtrate layers were separated, then the aqueous layer was extracted once with toluene (75 mL). The layers were separated, and then the two toluene layers were combined and filtered through a 0.2 um filter. The filtrate was transferred to a reaction vessel pre-rinsed with toluene filtered through a 0.2 um filter. The mixture was distilled under ca 300 Torr until a pot volume of ca 75 mL was reached, and then brought to 60° C.

While holding the process at 60° C., n-heptane was added (144 mL). The process was held at 60° C. for 40 minutes. The batch was then cooled to 45° C. over 20 minutes. Once the batch temperature reached 45° C., spontaneous crystallization occurred. The batch was held at 45° C. for 1 hour, then cooled to 15° C. over 30 minutes and allowed to granulate overnight at this temperature (16 hours total).

The slurry was filtered, the filter cake was washed with n-heptane (20 mL), and dried at 40° C., 20″ Hg, with no nitrogen bleed, for three days to isolate 82% of varenicline free base.

### B. Seeded Process

Varenicline tartrate (4.92 g) was dissolved in water (25 mL), then toluene (85 mL) was added. The mixture was heated to approx. 38° C., then 50% NaOH (w/w) (2.43 g) was added. After 1.5 hours, a slurry of activated carbon (0.25 g) in toluene (1.75 mL) was charged. The mixture stirred for 1.5 hours, then was filtered through a filter cake of diatomaceous earth. The filter cake was washed with toluene (7.5 mL).

The filtrate layers were separated, then the aqueous layer was extracted once with toluene (25 mL). The layers were separated, and then the two toluene layers were combined and filtered through a 0.2 um filter. This filtrate was transferred to a reaction vessel pre-rinsed with toluene filtered through a 0.2 um filter. The mixture was distilled under vacuum until a pot volume of ca 25 mL was reached. The mixture was returned to atmospheric pressure and brought to 60° C.

While holding the process at 60° C., n-heptane was added (48 mL) over 10 minutes. The process was held at 60° C. for 20 minutes. Varenicline free base form C was added as seed (30 mg, 0.6 wt %) and the process was held for 10 minutes. The batch was cooled to 50° C., held for one hour at 50° C., then was cooled to 15° C. over 70 minutes. The mixture granulated 15 hours, then was filtered. The filter cake was washed with n-heptane (10 mL) and dried at 60-65° C. under 17″ Hg with a nitrogen bleed for 22 hours. An 80% yield of product was isolated.

### Purity Data:

| | Unseeded Process | Seeded Process |
|---|---|---|
| HPLC potency | 98.4% | 100.0% |
| HPLC purity | 99.81% API | 100.0% API |
| | 0.18% toluene | |
| | 0.01% unknown | |
| XRPD | Form C match | Form C match |
| TGA | 1.693% wt loss | 0.513% wt loss |
| | between 30-143.5° C. | between 30-143.5° C. |
| Residual | 0.62% | 0.03% |
| toluene | | |
| Residual | 0.84% | 0.02% |
| heptane | | |

Other suitable solvents that could be suitable for this process are non-chlorinated solvents or solvent combinations selected from the group consisting of toluene, xylenes, hexanes, cyclohexanes, heptanes, n-heptane, octanes, nonanes and decanes.

The seeding process is preferred to produce a smaller range of particle size of varenicline free base Form C. A preferred particle size range is 100 to 250 microns. More preferred is 50 to 150 microns, and most preferred is 25 to 100 microns.

The above process produces substantially pure varenicline free base Form C suitable for administration to a human subject. By “substantially pure” it is meant that the varenicline free base Form C produced contains preferrably less than 5% by weight of N-formylvarenicline, relative to the total weight of varenicline and less than 5% by weight of N-carboxyvarenicline adduct, relative to the total weight of varenicline. More preferably, less than than 2% by weight of N-formylvarenicline, relative to the total weight of varenicline and less than 2% by weight of N-carboxyvarenicline adduct, relative to the total weight of varenicline is formed. Most preferrably less than 1% by weight of N-formylvarenicline, relative to the total weight of varenicline and less than 1% by weight of N-carboxyvarenicline adduct, relative to the total weight of varenicline is formed via the above process.

### Method B

200 Mg of compound IV (varenicline free base) was dissolved in a solvent selected from methylene chloride, isopropyl alcohol, methanol, and water. Once complete dissolution was visually verified, the solution was evaporated under reduced pressure to dryness. The resulting crystalline solid was allowed to dry under reduced pressure at 45-50° C. for three days.

Form C is determined to have a monoclinic crystal system with a P2(1)/n space group. The cell parameters at room temperature are: a=10.086 Å, b=10.258 Å, c=10.423 Å, α=90.00°, β=99.68°, γ=90.00°, cell volume=1063.03 Å³.

### Form D

A crystal of compound IV Form E was mounted for single crystal analysis and cooled to approximately −150° C. (15 g) was dissolved in water (75 mL), then toluene (255 mL) was added. The mixture was heated to approx. 38° C., then 50% NaOH (w/w) (7.29 g) was added. After 1.5 hours, the mixture was treated with a slurry of activated carbon (0.75 g) in toluene (5 mL), and then filtered. The filter cake was washed with toluene (22.5 mL).

### Form E

Compound IV (50 mg) and methyl tert-butyl ether saturated with water (3.5 mL) were added to a polypropylene reaction vessel. The mixture was heated to approximately 40° C. at approximately 1° C./minute, held at 40° C. for ten minutes, then cooled to −25° C. at approximately 3° C./minute. The system was held at −25° C. overnight. The system was heated to 5° C. at approximately 3° C./minute and then filtered. The filter cake isolated and stored in a sealed glass vial at 5° C.

Solids of compound IV (varenicline free base Form A, Form C, and Form E) were characterized by powder X-ray diffraction on a Siemens D5000 diffractometer as above. Solids of compound IV (varenicline free base, Form D) were characterized by single crystal X-ray diffraction and the powder X-ray diffraction pattern was calculated from single crystal data.

Table 9 lists the 2θ and relative intensities of all peaks that have a relative intensity of approximately >5% between 3 and 40°2θ in the sample for Form A of compound IV.

TABLE 9

| | Relativity |
|---|---|
| 2-theta | Intensity* |
| (degrees) | (%) |
| 7.9 | 6.8 |
| 8.5 | 41.4 |
| 8.8 | 20.2 |
| 11.5 | 9.7 |
| 15.4 | 26.4 |
| 15.9 | 13.4 |
| 16.2 | 14.9 |
| 17.1 | 100 |
| 17.8 | 43.2 |
| 19.0 | 34.4 |
| 20.5 | 11.3 |
| 21.0 | 13.1 |
| 22.0 | 12.1 |
| 22.9 | 11.9 |
| 24.0 | 12.2 |
| 24.3 | 15.2 |
| 25.4 | 14.2 |
| 26.1 | 7.8 |
| 26.7 | 8.1 |
| 27.6 | 7.5 |
| 29.4 | 10.8 |
| 30.4 | 6.8 |
| 30.8 | 7.2 |
| 31.7 | 6.8 |
| 32.8 | 7.2 |
| 34.7 | 6.8 |
| 37.4 | 5.4 |
| 39.6 | 5.4 |

*The relative intensity may vary depending on particle size and shape.

Table 10 lists the 2θ and relative intensities of all peaks that have a relative intensity of approximately >3% between 3 and 40°2θ in the sample for Form C of compound IV.

TABLE 10

| | Relativity |
|---|---|
| 2-theta | Intensity* |
| (degrees) | (%) |
| 11.3 | 100.0 |
| 12.2 | 13.4 |
| 13.4 | 8.2 |
| 14.2 | 10.5 |
| 16.0 | 6.8 |
| 17.3 | 40.0 |
| 19.4 | 84.3 |
| 19.8 | 29.4 |
| 20.4 | 58.0 |
| 20.6 | 73.5 |
| 22.0 | 65.5 |
| 22.5 | 6.4 |
| 22.8 | 6.8 |
| 24.4 | 3.7 |
| 25.4 | 4.7 |
| 26.9 | 30.8 |
| 27.2 | 4.6 |
| 27.4 | 16.4 |
| 28.3 | 13.0 |
| 29.4 | 12.2 |
| 31.4 | 4.4 |
| 31.8 | 5.3 |
| 32.2 | 5.8 |
| 33.8 | 10.0 |
| 34.6 | 4.7 |
| 34.9 | 4.2 |
| 36.1 | 6.1 |
| 37.3 | 19.5 |
| 39.6 | 3.2 |

*The relative intensity may vary depending on particle size and shape.

Table 11 lists the 2θ and relative intensities of all peaks that have a relative intensity of approximately >2% between 3 and 40°2θ in the sample for Form D of compound IV (varenicline free base).

TABLE 11

| | Relativity |
|---|---|
| 2-theta | Intensity* |
| (degrees) | (%) |
| 5.5 | 76.3 |
| 6.9 | 25.5 |
| 7.6 | 23.6 |
| 9.9 | 54.3 |
| 11.1 | 58.6 |
| 11.4 | 100.0 |
| 13.1 | 6.3 |
| 13.4 | 19.3 |
| 13.8 | 8.4 |
| 14.5 | 77.0 |
| 15.3 | 64.7 |
| 15.5 | 33.5 |
| 15.9 | 14.6 |
| 16.1 | 13.9 |
| 16.3 | 13.9 |
| 16.7 | 20.3 |
| 17.5 | 22.1 |
| 18.0 | 6.6 |
| 18.8 | 44.8 |
| 19.0 | 54.8 |
| 19.7 | 10.5 |
| 20.0 | 13.8 |
| 20.8 | 16.7 |
| 21.2 | 25.2 |
| 21.6 | 8.2 |
| 21.7 | 7.6 |
| 22.4 | 6.0 |
| 23.0 | 11.5 |
| 23.2 | 12.5 |
| 23.7 | 20.6 |
| 24.3 | 8.2 |
| 24.5 | 9.9 |
| 24.9 | 12.9 |
| 25.2 | 5.7 |
| 25.6 | 4.3 |
| 26.0 | 15.9 |
| 26.5 | 32.7 |
| 26.7 | 53.4 |
| 27.1 | 89.5 |
| 27.8 | 20.7 |
| 28.1 | 15.1 |
| 28.4 | 50.3 |
| 29.2 | 8.1 |
| 29.6 | 18.1 |
| 29.9 | 48.7 |
| 30.3 | 38.3 |
| 30.6 | 15.5 |
| 31.1 | 23.6 |
| 31.9 | 2.2 |
| 32.3 | 8.3 |
| 32.6 | 4.4 |
| 33.0 | 6.8 |
| 33.3 | 12.5 |
| 33.9 | 4.7 |
| 34.3 | 8.6 |
| 34.5 | 6.4 |
| 35.1 | 6.9 |
| 36.4 | 9.4 |
| 36.8 | 3.2 |
| 37.0 | 2.8 |
| 37.2 | 2.4 |
| 37.6 | 4.0 |
| 37.9 | 3.8 |
| 38.2 | 2.9 |
| 38.9 | 8.3 |
| 39.4 | 6.2 |

*The relative intensity may vary depending on particle size and shape.

Table 12 lists the 2θ and relative intensities of all peaks that have a relative intensity of approximately >0.5% between 3 and 40°2θ in the sample for Form E compound IV (varenicline free base).

TABLE 12

| | Relativity |
|---|---|
| 2-theta | Intensity* |
| (degrees) | (%) |
| 5.5 | 38.4 |
| 6.8 | 2.1 |
| 7.5 | 9.4 |
| 7.8 | 1.1 |
| 9.1 | 3.5 |
| 9.8 | 4.4 |
| 11.0 | 44.8 |
| 11.3 | 100.0 |
| 13.3 | 2.9 |
| 13.6 | 0.9 |
| 14.4 | 8.8 |
| 15.1 | 27.4 |
| 16.1 | 13.0 |
| 16.5 | 20.1 |
| 17.2 | 1.0 |
| 18.5 | 3.3 |
| 18.8 | 1.4 |
| 19.5 | 2.7 |
| 20.5 | 3.6 |
| 20.9 | 1.6 |
| 21.3 | 5.7 |
| 22.2 | 2.7 |
| 22.7 | 11.1 |
| 23.6 | 1.4 |
| 24.1 | 0.9 |
| 24.7 | 0.7 |
| 25.9 | 1.6 |
| 26.7 | 6.5 |
| 27.5 | 3.4 |
| 27.8 | 3.6 |
| 28.1 | 6.3 |
| 29.1 | 1.0 |
| 29.5 | 1.1 |
| 29.9 | 1.8 |
| 30.4 | 4.4 |
| 32.2 | 1.4 |
| 32.6 | 5.7 |
| 33.0 | 1.5 |
| 33.5 | 1.3 |
| 34.3 | 1.3 |
| 34.8 | 1.1 |
| 36.1 | 1.2 |
| 37.4 | 1.4 |
| 38.4 | 1.1 |
| 38.9 | 0.9 |
| 39.3 | 1.8 |
| 39.8 | 1.0 |

*The relative intensity may vary depending on particle size and shape.

Compound IV of the present invention may exist in anhydrous forms as well as hydrated and solvated forms and are intended to be encompassed within the scope of the present invention. Table 13 shows the unique identifying peak sets (±0.2°2θ) for each of the crystal forms of Compound IV.

TABLE 13

| Form A | Form C | Form D | Form E |
|---|---|---|---|
| 2-theta | 2-theta | 2-theta | 2-theta |
| (degrees) | (degrees) | (degrees) | (degrees) |
| 8.5 | 11.3 | 5.5 | 5.5 |
| 8.8 | 17.3 | 9.9 | 9.1 |
| 15.4 | 19.8 | 15.3 | 9.8 |
| 17.1 | 20.6 | 17.5 | 16.5 |
| 19.0 | 22.0 | 27.1 | 22.7 |

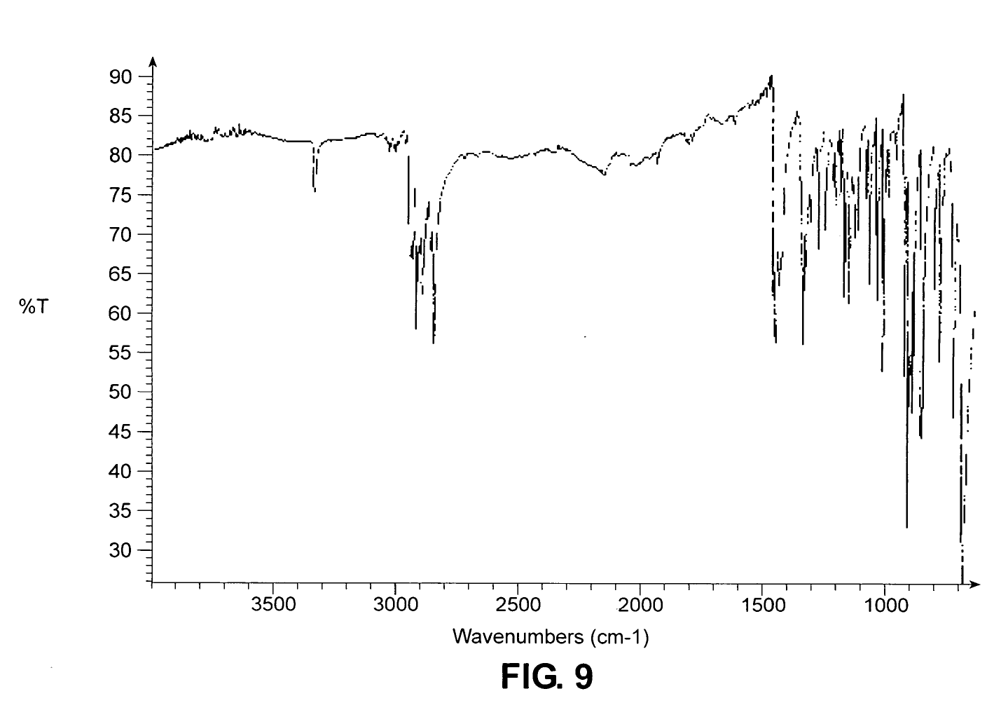

Solids of compound IV (varenicline free base Form C) were characterized by infrared spectroscopy using an IlluminatIR™ Fourier transform infrared (FT-IR) microspectrometer (SensIR Technologies) equipped with a 10 volt ceramic IR source, a potassium bromide (KBr) beamsplitter, and a mercury-cadmium-telluride (MCT) detector. A diamond attenuated total reflectance (ATR) objective (ContactIR, SensIR Technologies) was used for data acquisition. Each spectrum represents 100 co-added scans using a 100 μm masking aperture collected at a spectral resolution of 4 cm-1, using Happ-Genzel apodization. Sample preparation consisted of placing the sample on a standard glass microscope slide under ambient conditions. A background spectrum was first acquired using the diamond attenuated total reflectance (ATR) objective. Spectra were acquired for three different regions of each sample to ensure adequate sampling. The displayed spectra result from the arithmetic mean of the three individual spectra. Peaks were identified using the ThermoNicolet Omnic version 7.3 software peak picking algorithm using a sensitivity setting of 85 and an intensity threshold of 90.0 for the region 650-1900 cm⁻¹ and a sensitivity setting of 85 and an intensity threshold of 82.8 for the region 2400-3400 cm⁻¹. Typically, the error associated with this instrument method is ±4 cm⁻¹. Diamond spectral features in the region between 2400-1900 cm-1 are present in all FT-ATR spectra (Ferrer, N.; Nogués-Carulla, J. M. Diamond and Related Materials 1996, 5, 598-602. Thongnopkun, P.; Ekgasit, S. Diamond and Related Materials 2005, 14, 1592-1599. The FT-IR spectrum of Compound IV Form C is provided in FIG. 9.

TABLE 14
Wavenumber (cm⁻¹)

693
733
765
791
814
861
867
903
913
921
939
974
1007
1029
1053
1085
1096
1128
1139
1165
1185
1202
1221
1234
1263
1292
1324
1341
1353
1385
1449
1464
1472
1488
1506
1520
1539
1558
1574
1633
1684
1803
1821
2445
2539
2577
2676
2729
2852
2869
2899
2923
2942
2950
2972
3013
3035
3044
3055
3085
3342

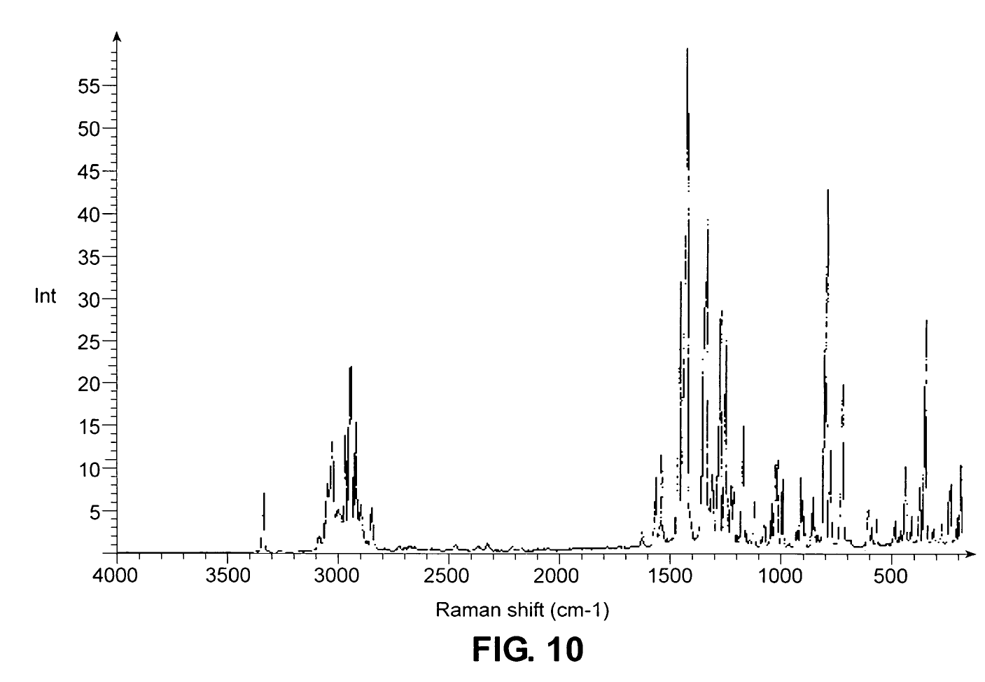

Solids of compound IV (varenicline free base) Form C were characterized by Raman spectroscopy using a ThermoNicolet 960 FT-Raman spectrometer equipped with a 1064 nm NdYAG laser and InGaAs detector. Prior to data acquisition, instrument performance and calibration verifications were conducted using polystyrene. Samples were analyzed in glass NMR tubes. The spectra were collected using 0.5 W of laser power and 100 co-added scans. All spectra were recorded using 2 cm-1 resolution and Happ-Genzel apodization. Four spectra were recorded for each sample, with 45° sample rotation between spectral collections. The spectra for each sample were averaged together, and then intensity normalization was performed prior to peak picking. Peaks were identified using the ThermoNicolet Omnic 7.3 software peak picking algorithm. Peak picking for compound IV Form C was first performed for the 2800-3400 cm-1 region using intensity threshold of 0.008 and a sensitivity of 75. Subsequently, peak picking was performed for the 100-1700 cm-1 region using an intensity threshold of 0.017 and a sensitivity of 88. With this method, the positional accuracy of these peaks is ±2 cm-1. The FT-Raman spectrum of compound IV Form C is provided in FIG. 10.

TABLE 15
Wavenumber (cm⁻¹)

115
130
195
213
243
248
283
320
358
381
420
449
471
493
577
597
617
736
791
814
849
859
868
912
921
940
975
1004
1029
1038
1054
1085
1097
1132
1154
1169
1185
1204
1225
1236
1243
1266
1289
1323
1355
1362
1446
1471
1523
1549
1573
1634
2854
2873
2903
2925
2949
2955
2973
3007
3029
3035
3055
3087
3343

Solids of compound IV (varenicline free base) Form C were characterized by Solid-state Nuclear Resonance Spectroscopy at ambient temperature and pressure on a Bruker-Biospin 4 mm BL CPMAS probe positioned into a wide-bore Bruker-Biospin Avance DSX 500 MHz NMR spectrometer. Approximately 80 mg of sample was tightly packed into a 4 mm ZrO₂ spinner and the sample was positioned at the magic angle and spun at 15.0 kHz. The fast spinning speed minimized the intensities of the spinning side bands. The number of scans was adjusted to obtain adequate S/N.

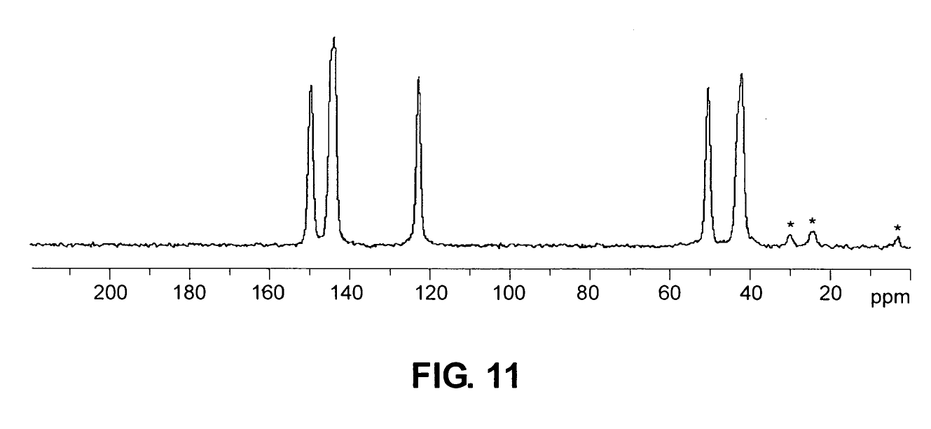

The ¹³C solid state spectrum was collected using a proton decoupled cross-polarization magic angle spinning experiment (CPMAS; Table 16). The cross-polarization contact time was set to 2.0 ms. A proton decoupling field of approximately 90 kHz was applied. 480 scans were collected. The recycle delay was adjusted to 380 seconds. The spectrum was referenced using an external standard of crystalline adamantane, setting its upfield resonance to 29.5 ppm. Typically, the error associated with this instrument method is ±0.2 ppm. The ¹³C CPMAS spectra of Compound IV Form C is provided in FIG. 11. Spinning sidebands are noted with an asterisk.

TABLE 16

| ¹³C Chemical | |
|---|---|
| Shifts^{a} [ppm] | Intensity^{b} |
| 149.8 | 9.2 |
| 144.6 | 11.2 |
| 143.9 | 12.0 |
| 122.9 | 9.7 |
| 50.8 | 9.2 |
| 43.3^{c} | — |
| 42.5 | 9.9 |

^{a}Referenced to external sample of solid phase adamantane at 29.5 ppm.
^{b}Defined as peak heights. Intensities can vary depending on the actual setup of the CPMAS experimental parameters and the thermal history of the sample. CPMAS intensities are not necessarily quantitative.
^{c}Peak shoulder.

Compound IV Form C, produced using the process described in this specification, can contain a N-carboxyvarenicline adduct and a N-formyl adduct of Compound IV. The N-carboxy adduct of Compound IV is of the structure

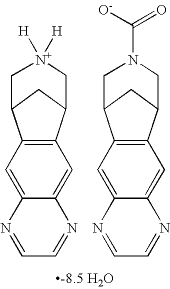

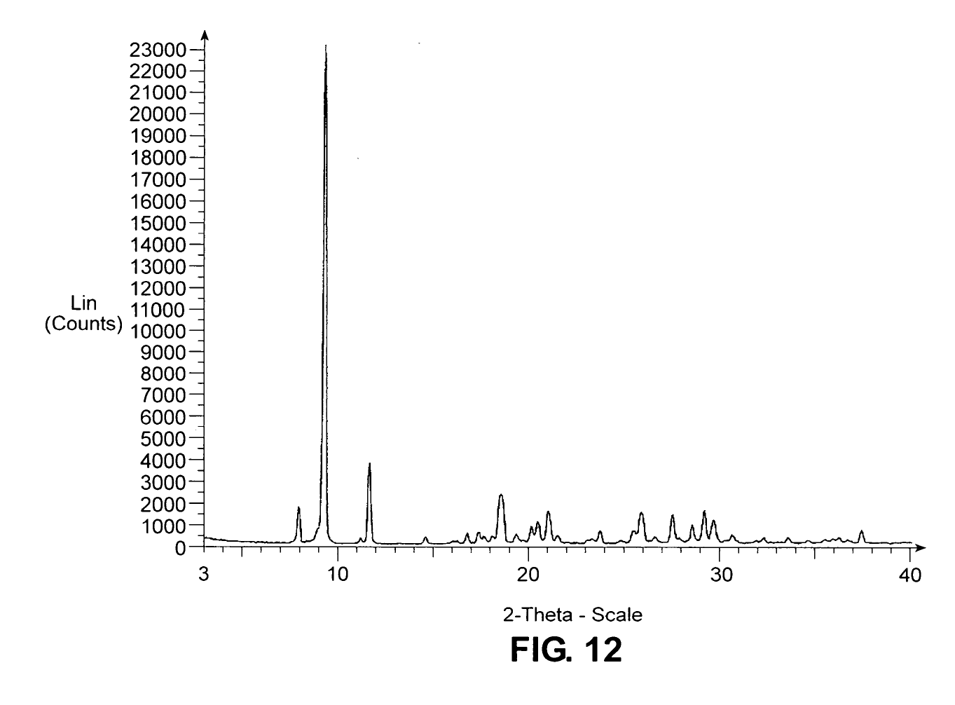

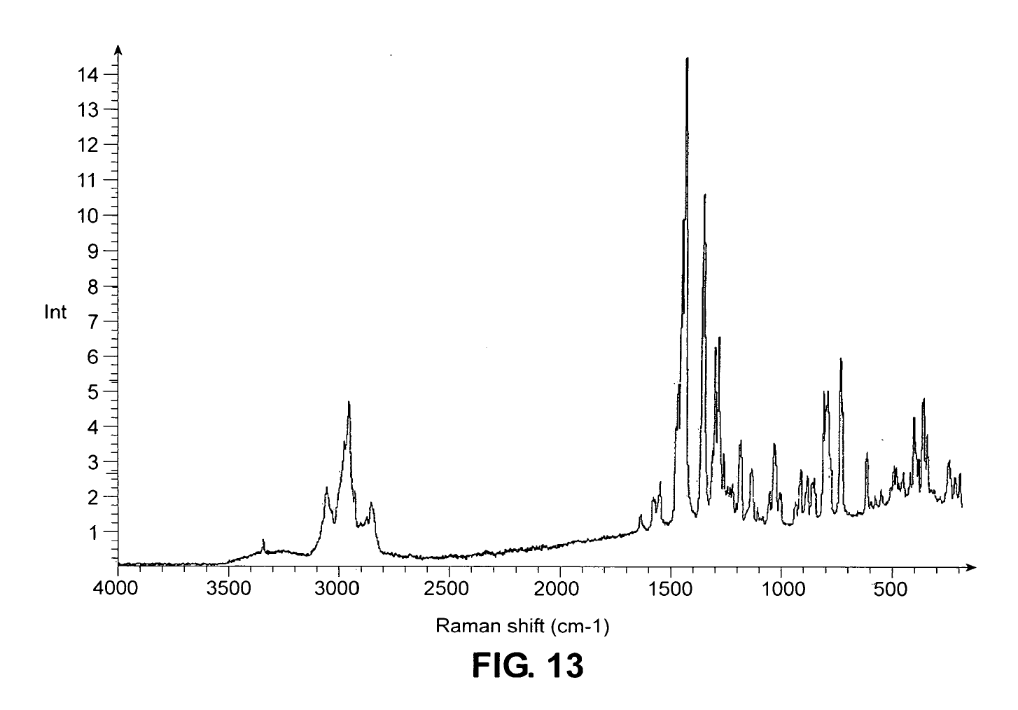

and is observed when Form C is stored at high humidities. The known crystal form of the N-carboxyvarenicline adduct exhibits the X-ray powder diffraction pattern provided in FIG. 12 and the Raman spectrum is provided in FIG. 13. The lot used to generate this X-ray powder diffraction and Raman data may contain residual compound IV Form C.

The N-formylvarenicline adduct is of the structure

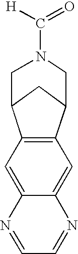

and is observed in the mother liquor of the crystallization process described in the specification. It can be detected by HPLC using the following conditions:
aqueous buffer 0.1% H3PO4, 5 mM OSA in water:Methanol (66:34, v/v)
Agilent Zorbax SB-C18 column, 150 mm length×4.6 mm I.D.
Column temperature—50 degrees Celsius; UV detection (210 nm
1.5 mL/min flow rate using a 5 micro liter injection volume.

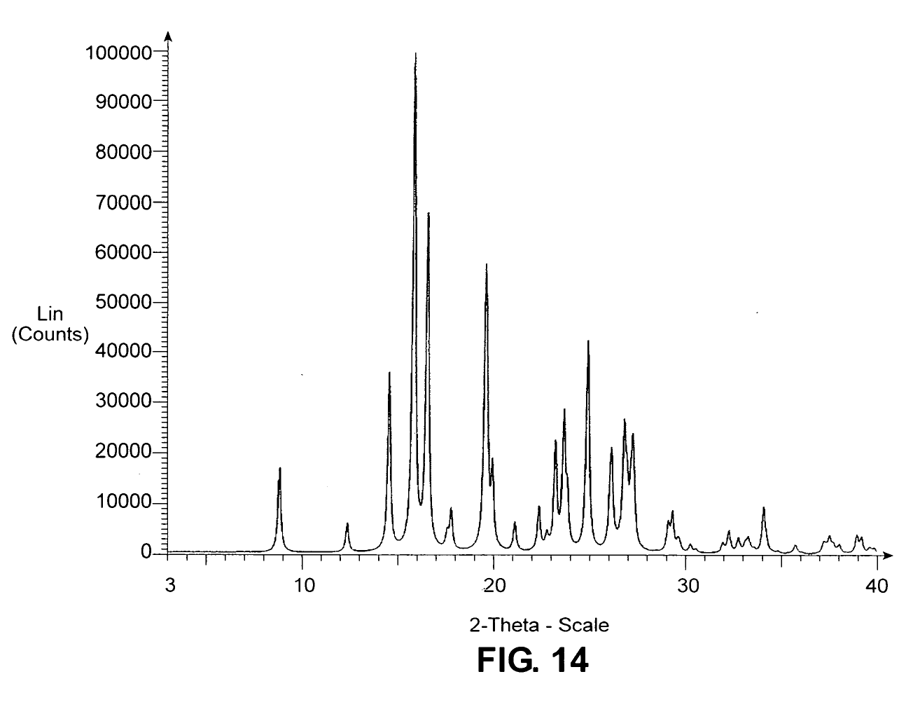

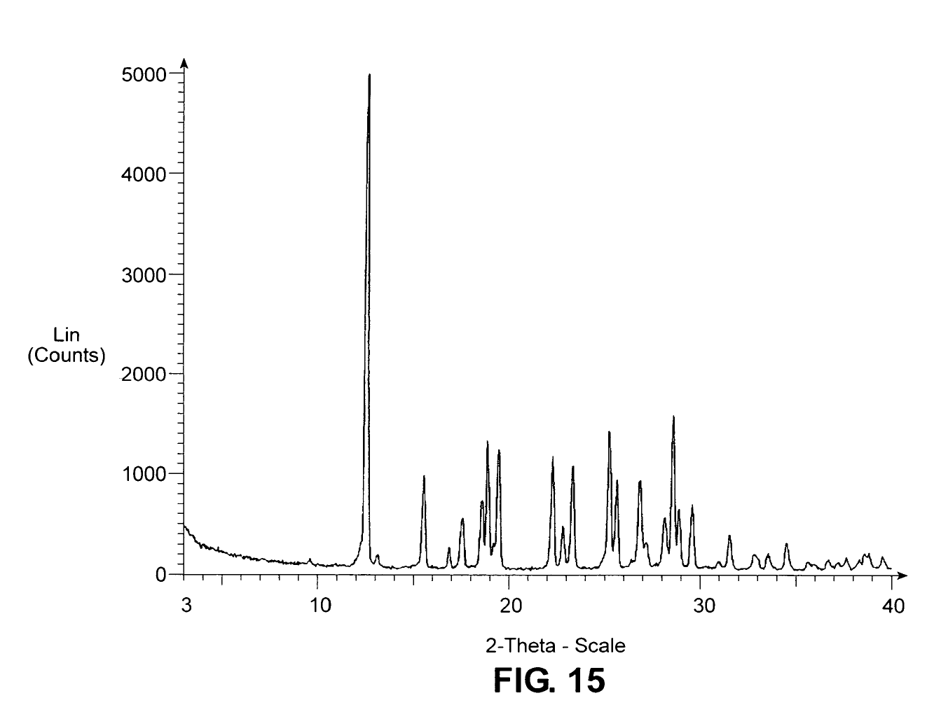

The N-formylvarenicline adduct is a known compound and has been disclosed in United States Patent Application Publication Number 2004/0235850. The known crystal form of the N-formyl adduct exhibits an X-ray powder diffraction pattern consistent with the calculated pattern provided in FIG. 14.

Solids of the N-carboxyvarenicline adduct were characterized by powder X-ray diffraction on a Siemens D5000 diffractometer as above. These solids may contain residual compound IV Form C. Solids of the N-formylvarenicline adduct were characterized by single crystal X-ray diffraction and the powder X-ray diffraction pattern was calculated from single crystal data.

Table 17 lists the 2θ and relative intensities of all peaks that have a relative intensity of approximately >0.5% between 3 and 40°2θ in the sample of the N-carboxyvarenicline adduct. This sample may contain residual compound IV Form C.

TABLE 17

| | Relativity |
|---|---|
| 2-theta | Intensity* |
| (degrees) | (%) |
| 7.9 | 7.8 |
| 9.2 | 100.0 |
| 11.1 | 1.7 |
| 11.6 | 16.3 |
| 14.5 | 1.8 |
| 16.0 | 1.1 |
| 16.8 | 2.7 |
| 17.3 | 2.9 |
| 17.6 | 2.0 |
| 18.0 | 2.1 |
| 18.5 | 10.3 |
| 19.3 | 2.5 |
| 19.7 | 1.3 |
| 20.1 | 4.0 |
| 20.5 | 5.0 |
| 21.0 | 7.0 |
| 21.5 | 2.1 |
| 23.0 | 1.4 |
| 23.3 | 1.5 |
| 23.7 | 3.2 |
| 24.8 | 1.2 |
| 25.4 | 3.2 |
| 25.8 | 6.9 |
| 26.5 | 2.0 |
| 27.5 | 6.5 |
| 27.8 | 1.7 |
| 28.5 | 4.4 |
| 29.1 | 7.3 |
| 29.6 | 5.4 |
| 30.2 | 1.4 |
| 30.6 | 2.5 |
| 32.2 | 1.8 |
| 33.5 | 1.9 |
| 34.5 | 1.4 |
| 35.4 | 1.5 |
| 35.8 | 1.7 |
| 36.2 | 2.0 |
| 36.6 | 1.5 |
| 37.3 | 3.4 |

The relative intensity may vary depending on particle size and shape.

Table 18 lists the 2θ and relative intensities of all peaks that have a relative intensity of approximately >0.5% between 3 and 40°2θ in the sample for the N-formylvarenicline adduct of Compound IV.

TABLE 18

| | Relativity |
|---|---|
| 2-theta | Intensity* |
| (degrees) | (%) |
| 8.8 | 16.9 |
| 12.4 | 6.1 |
| 14.6 | 35.9 |
| 15.8 | 100.0 |
| 16.5 | 68.2 |
| 17.6 | 5.3 |
| 17.8 | 9.1 |
| 19.6 | 57.8 |
| 19.9 | 18.9 |
| 21.2 | 6.4 |
| 22.4 | 9.5 |
| 22.8 | 4.8 |
| 23.3 | 22.5 |
| 23.7 | 28.6 |
| 24.9 | 42.4 |
| 26.2 | 21.1 |
| 26.9 | 26.7 |
| 27.3 | 23.9 |
| 29.2 | 6.8 |
| 29.3 | 8.6 |
| 29.6 | 3.5 |
| 30.3 | 2.0 |
| 30.5 | 1.0 |
| 32.0 | 2.4 |
| 32.3 | 4.8 |
| 32.8 | 3.3 |
| 33.3 | 3.5 |
| 34.1 | 9.4 |
| 34.8 | 0.7 |
| 35.8 | 1.9 |
| 37.3 | 2.7 |
| 37.5 | 3.8 |
| 38.0 | 2.0 |
| 39.0 | 3.9 |
| 39.2 | 3.5 |
| 39.7 | 1.4 |

The relative intensity may vary depending on particle size and shape.

Table 19 shows the unique sets of identifying X-ray powder diffraction reflections for the N-carboxyvarenicline adduct and N-formylvarenicline.

TABLE 19

| N-carboxyvarenicline | |
|---|---|
| adduct | N-formylvarenicline |
| 2-theta | 2-theta |
| (degrees) | (degrees) |
| 7.9 | 8.8 |
| 9.2 | 12.4 |
| 11.6 | 16.5 |
| 16.8 | 17.8 |
| 18.5 | 19.6 |

Solids of the N-carboxyvarenicline adduct were characterized by Raman spectroscopy on a ThermoNicolet 960 FT-Raman spectrometer equipped with a 1064 nm NdYAG laser and InGaAs detector as above (Table 20). These solids may contain residual compound IV Form C. Peak picking for the N-carboxyvarenicline adduct was first performed for the 2800-3400 cm-1 region using an intensity threshold of 0.045 and a sensitivity of 70. Subsequently, peak picking was performed for the 100-1700 cm-1 region using an intensity threshold of 0.051 and a sensitivity of 81. With this method, the positional accuracy of these peaks is ±2 cm-1.

TABLE 20
Wavenumber (cm⁻¹)

134
160
196
218
245
284
311
343
359
364
382
402
420
450
470
487
494
550
577
597
616
658
730
738
796
814
854
866
887
915
920
937
975
1005
1012
1030
1038
1056
1085
1111
1139
1187
1209
1225
1236
1248
1266
1290
1306
1316
1361
1446
1458
1472
1483
1528
1550
1574
1581
1637
2854
2871
2900
2926
2955
2973
3031
3054
3344

Table 21 shows unique FT-Raman bands for the N-carboxyvarenicline adduct that can be used to differentiate the N-carboxyvarenicline adduct from compound IV Form C.

TABLE 21
Wavenumber (cm⁻¹)

343
402
550
887
1012
1458

All of the above crystalline forms and mixtures thereof may be effectively utilized in the process scheme described above in all the various permutations and combinations. The various crystalline forms may be utilized as both intermediaries or final products as applicable for the specific application. In such final form, compound IV has utility for use in a transdermal patch as a means for medicinal introduction on an extended basis.

### Example 1

### Matrix Type Transdermal Patch

Varenicline free base form C is mixed with the aqueous dispersion of NACOR 72-9965 (hydrophobic acrylic copolymer from National Starch) to achieve a 2% (w/w) concentration of active ingredient in the dried film after film casting. The adhesive mixture is cast on a release coated polymer film (Rexam Release Technologies; W. Chicago, Ill.) and is dried at 60° C. in a convective oven and cut to achieve a 2 mgA dose of the active ingredient. The dried film is laminated to a polyester film laminate (SCOTCHPACK #1012, 3M Pharmaceuticals; St. Paul, Minn.).

### Example 2

### Matrix Type Transdermal Patch Systems

(1) Varenicline free base form C is dissolved or dispersed in a polyacrylate solution, such as Duro-Tak® 387-2052 adhesive. Appropriate solvent, enhancer and/or filler is added in the adhesive dispersion, and mixed well. Air is removed from the resulting mixture and laminated on a release liner, such as Medirelease® 2228, to form a coating thickness of 0.5-2 mm. The adhesive layer is dried at room temperature for 5-10 min and then at 40-80° C. for 15-30 min to remove all volatile solvents. A backing sheet, such as Mediflex® 1200, is coated on the adhesive side. The resulting patches of a desired size are stored in sealed packages.

(2) Varenicline free base form C is dissolved or dispersed in a polyisobutylene (PIB) based adhesive, such as Duro-Tak® 87-6173. The following procedures are similar to those described in the previous section.

(3) Varenicline free base form C is dissolved or dispersed in a silicone-based adhesive, such as Bio-PSA® 7-4302. The following procedures are similar to those described in the previous section.

1. Substantially pure varenicline free base Form C suitable for administration to a human subject comprising
a) less than 2% by weight of a first impurity N-formylvarenicline, relative to the total weight of varenicline, and
b) less than 2% by weight of a second impurity N-carboxyvarenicline adduct, relative to the total weight of varenicline,
wherein Form C is characterized by a powder x-ray diffraction pattern obtained using CuK α radiation which includes peaks at 2θ (degrees) 11.3, 17.3, 19.8, 20.6, and 22.0±0.2.

2. Substantially pure varenicline free base Form C suitable for administration to a human subject comprising
a) less than 2% by weight of a first impurity N-formylvarenicline, relative to the total weight of varenicline, and
b) less than 2% by weight of a second impurity N-carboxyvarenicline adduct, relative to the total weight of varenicline,
wherein Form C is characterized by a ¹³C proton decoupled cross-polarization magic angle
spinning (CPMAS) solid state NMR spectra which includes peaks at 149.8, 144.6, 143.9, 122.9, 50.8, and 42.5±0.2 ppm referenced to an external sample of solid phase adamantane at 29.5 ppm.

3. Substantially pure varenicline free base Form C according to claim 1, comprising
a) less than 1% by weight of a first impurity N-formylvarenicline, relative to the total weight of varenicline, and
b) less than 1% by weight of a second impurity N-carboxyvarenicline adduct, relative to the total weight of varenicline.

4. A composition including substantially pure varenicline free base Form C as defined in claim 1.

5. The composition of claim 4 wherein said composition comprises a transdermal patch and wherein the substantially pure varenicline free base Form C is a dispersed particulate suspension.

6. A process to form substantially pure varenicline free base Form C as defined in claim 1, comprising the step of crystallizing varenicline from the crystallization solvent or solvent combination comprising an organic non-chlorinated solvent.

7. The process according to claim 6 wherein said non-chlorinated solvent or solvent combination is selected from the group consisting of toluene, xylenes, hexanes, cyclohexanes, heptanes, n-heptane, octanes, nonanes and decanes.

8. The process according to claim 7 wherein the solvent or solvent combination is toluene and n-heptane.

9. The process according to claim 6 further comprising the use of seeding to prepare smaller sized particles of substantially pure varenicline free base Form C.

10. A composition including substantially pure varenicline free base Form C as defined in claim 3.

11. The composition of claim 10, wherein said composition comprises a transdermal patch and wherein the substantially pure varenicline free base Form C is a dispersed particulate suspension.

12. A process to form substantially pure varenicline free base Form C as defined in claim 3, comprising the step of crystallizing varenicline from the crystallization solvent or solvent combination comprising an organic non-chlorinated solvent.

13. The process according to claim 12, wherein said non-chlorinated solvent or solvent combination is selected from the group consisting of toluene, xylenes, hexanes, cyclohexanes, heptanes, n-heptane, octanes, nonanes and decanes.

14. The process according to claim 13, wherein the solvent or solvent combination is toluene and n-heptane.

15. The process according to claim 14, further comprising the use of seeding to prepare smaller sized particles of substantially pure varenicline free base Form C.
